(19) United States
(12) Patent Application Publication
(10) Pub. No.: US 2024/0376555 A1
(43) Pub. Date: Nov. 14, 2024

(54) TNRA MODIFICATION-SENSITIVE CODON TRANSLATION ASSAY COMPRISING LUMINESCENCE-BASED REPORTER SYSTEM

(71) Applicant: Gesval S.A., Angleur (BE)

(72) Inventors: Pierre Close, Angleur (BE); Francesca Rapina, Liege (BE); Alain Chariot, Neupre (BE)

(21) Appl. No.: 18/284,924

(22) PCT Filed: Feb. 27, 2022
(86) PCT No.: PCT/EP2022/054902

§ 371 Date: Sep. 29, 2023

(30) Foreign Application Priority Data
Mar. 29, 2021 (BE) BE2021/5237

(51) Int. Cl. C12Q 1/6897

(52) U.S. Cl. CPC C12Q 1/6897

(57) ABSTRACT

An assay using stably transformed human cell lines comprising repetitions of codons sensitive to tRNA modifications upstream of a first luminescence-based reporter gene and repetitions of their synonymous codons upstream of a second luminescence-based reporter gene is helpful in rapidly identifying modulators of tRNA modifying enzymes.

### BACKGROUND

Cells need to constantly change their proteome in order to respond to external and internal stimuli. This continuous adaptation is regulated by changes in transcription and translation of mRNAs [1]-[3]. Transfer RNAs (tRNAs) are the most abundant non-coding RNA molecules in cells: their role is to decode mRNAs through the correct pairing of mRNA codons with tRNA anticodon within the ribosome.

The genomic code is of degenerative nature. 61 mRNA codons code for 20 amino acids. Different genes encode sequence variants of tRNAs recognizing the same mRNA codon. Consequently, tRNAs repertoires are extremely heterogeneous among tissues and cellular populations [4]. The tissue specificity of tRNA pools is furthermore increased by the presence of a variety of tRNA modification patterns [5]. Initially, this tissue-specific expression of tRNA pools was considered void of functional relevance. However, breast cancers cells expressed significantly increased amounts of specific tRNAs compared to healthy breast lines [7]. This specific overexpression was causative of breast metastatic potential [8].

Amino acids are coded in mRNAs. The use of synonymous codons plays a critical role in protein expression and protein folding [9]-[12].

Cancers code for the amino acids differently from healthy tissues. This cancer-specific codon usage has an impact on cancer biology [8], [15].

The present inventors already demonstrated that certain modified tRNAs are responsible for the decoding fidelity of codons enriched in cancers. These modified tRNAs are

necessary to correctly establish onco-proteomes [13], [16], [17]. Consequently, tRNA

diversity has a key role in proteome rewiring in cancer [8], [18]-[20].

### SCIENTIFIC REFERENCES CITED

[1] R. Nagel, E. A. Semenova, and A. Berns, “Drugging the addict: non-oncogene addiction as a target for cancer therapy,” EMBO Rep., 2016.
[2] J. Chu, M. Cargnello, I. Topisirovic, and J. Pelletier, “Translation Initiation Factors: Reprogramming Protein Synthesis in Cancer,” Trends in Cell Biology. 2016.
[3] M. L. Truitt and D. Ruggero, “New frontiers in translational control of the cancer genome,” Nature Reviews Cancer. 2016.
[4] S. Kirchner and Z. Ignatova, “Emerging roles of tRNA in adaptive translation, signalling dynamics and disease,” Nature Reviews Genetics. 2015.
[5] P. Schimmel, “RNA Processing and Modifications: The emerging complexity of the tRNA world: Mammalian tRNAs beyond protein synthesis,” Nature Reviews Molecular Cell Biology. 2018.
[7] M. Pavon-Etemod, S. Gomes, R. Geslain, Q. Dai, M. R. Rosner, and T. Pan, “tRNA over-expression in breast cancer and functional consequences,” Nucleic Acids Res., 2009.
[8] H. Goodarzi, H. C. B. Nguyen, S. Zhang, B. D. Dill, H. Molina, and S. F. Tavazoie, “Modulated expression of specific tRNAs drives gene expression and cancer progression,” Cell, 2016.
[9] G. Hanson and J. Coller, “Translation and Protein Quality Control: Codon optimality, bias and usage in translation and mRNA decay,” Nature Reviews Molecular Cell Biology. 2018.
[10 ] E. M. Novoa and L. Ribas de Pouplana, “Speeding with control: Codon usage, tRNAs, and ribosomes,” Trends in Genetics. 2012.
[11] G. N. Jacobson and P. L. Clark, “Quality over quantity: Optimizing co-translational protein folding with non-'optimal' synonymous codons,” Current Opinion in Structural Biology. 2016.
[12] A. A. Komar, “The Yin and Yang of codon usage,” Human Molecular Genetics. 2016.
[13] F. Rapino et al., “Codon-specific translation reprogramming promotes resistance to targeted therapy,” Nature, 2018.
[14] F. Rapino, S. Delaunay, Z. Zhou, A. Chariot, and P. Close, “tRNA Modification: Is Cancer Having a Wobble?,” Trends in Cancer. 2017.
[15] S. Delaunay and M. Frye, “RNA modifications regulating cell fate in cancer,” Nature Cell Biology. 2019.
[16] A. Ladang et al., “Elp3 drives Wnt-dependent tumor initiation and regeneration in the intestine,” J. Exp. Med., 2015.
[17] S. Delaunay et al., “Elp3 links tRNA modification to IRES-dependent translation of LEF1 to sustain metastasis in breast cancer,” J. Exp. Med., 2016.
[18] H. Goodarzi, X. Liu, H. C. B. Nguyen, S. Zhang, L. Fish, and S. F. Tavazoie, “Endogenous tRNA-derived fragments suppress breast cancer progression via YBX1 displacement,” Cell, 2015.
[19] S. Blanco et al., “Stem cell function and stress response are controlled by protein synthesis,” Nature, 2016.
[20] S. Q. Huang et al., “The dysregulation of tRNAs and tRNA derivatives in cancer,” Journal of Experimental and Clinical Cancer Research. 2018.

### PRIOR ART

Cell-free assays to study the role of tRNA molecules have already been described.

WO2018081788A1 to the University of Cornell describes cell-free methods of enhancing translation ability and stability of RNA molecules, treatments and kits.

U.S. Pat. No. 10,921,326B2 to Anima biotech describes cell-free methods for labelling transfer RNA comprising replacing the uracil component of a dihydrouridine with a fluorophore as well as methods for assessing protein synthesis through such a labelled tRNA.

WO2017011766A1 to the University of Cornell discloses a cell-free method of enhancing the translation ability of an RNA molecule.

However, the above-described methods are either cell-free, complex or require substantial equipment and knowhow. Therefore, assessing the efficacy of modulators of tRNA modifying enzymes in a reliable, less costly but efficient way still is a challenge

### SHORT DESCRIPTION OF THE INVENTION

The inventors have surprisingly found that an assay using stably transformed human cell lines comprising repetitions of tRNA modification-sensitive codons upstream of a first luminescence-based reporter gene and repetitions of their synonymous codons upstream of a second luminescence-based reporter gene may be helpful in rapidly identifying modulators of tRNA modifying enzymes. Such modulators are hereinafter referred to as tRNA translation modification modulators.

In a preferred embodiment, the constructs are dual luciferase constructs.

In a further preferred embodiment, the constructs encode six in frame repetitions of a tRNA modification-sensitive codon, in particular a U₃₄ codon chosen from the group consisting of AAA, GAA and CAA upstream of the first luciferase reporter gene and six in frame repetitions of the corresponding synonymous codon, whose decoding does not require any tRNA modification, upstream of the second luciferase reporter gene.

Consequently, tRNA modification modulators impact the luminescence activity ratio of the first and of the second luminescence-based reporter gene and thus allow for tRNA modification modulator screening.

In a further preferred embodiment, the tRNA modification modulator is an inhibitor of cancer-specific codon-dependent mRNA translation. Accordingly, tRNA modification modulators detected by the assay of the present invention may be useful in controlling cancer development and acquired resistance.

Accordingly, a first aspect of the invention is an assay for detecting a modulator of a tRNA-related condition, comprising the steps of:
a. Providing one or more recombinant DNA constructs comprising
   i. A DNA oligonucleotide sequence encoding a first luminescence-based reporter gene operatively linked to a first promoter,
   ii. A DNA oligonucleotide sequence encoding a second luminescence-based reporter gene operatively linked to a second promoter;
b. Introducing the one or more recombinant DNA constructs of step a) into a system so as to allow for replication and translation in order to obtain a transduced system;
c. Exposing the transduced system of step b) to one or more candidate tRNA modification modulators to obtain a modulated system;
d. Inducing the luminescence emission of the modulated system of step c) to obtain an induced system;
e. Measuring the luminescence emission of the first and of the second luminescence-based reporter genes in the induced system of step d);
f. Correlating the signal measured in step e) to the one or more candidate tRNA modification modulators; and
g. Optionally selecting a modulator of a tRNA-related condition from the one or more candidate tRNA modification modulators;
wherein the recombinant DNA constructs comprise 3 to 10 in frame repetitions of a first codon upstream of the first luminescence-based reporter gene;
wherein the first codon is a tRNA modification-sensitive codon;
wherein the recombinant DNA constructs further comprises 3 to 10 in frame repetitions of a second codon upstream of the second luminescence-based reporter gene, and
wherein the second codon is the synonymous codon of the first codon.

In another embodiment, the tRNA modification-sensitive codon is selected from the group consisting of AAA, GAA, and CAA.

In another embodiment, the second codon is selected from the group consisting of AAG, GAG, and CAG.

In another embodiment, the recombinant DNA constructs comprise 4 to 8 repetitions of the first and of the second codon.

In another embodiment, the recombinant DNA constructs comprise 5 to 7 in frame repetitions of the first and of the second codon.

In another embodiment, the recombinant DNA constructs comprise 6 in frame repetitions of the target codons and of the corresponding synonymous codon.

In another embodiment, the first or the second reporter genes are fluorescence-based reporter genes.

In another embodiment, the luminescence-based based reporter genes are Nanoluc, Firefly; Nanoluc-PEST or Firefly-PEST.

In another embodiment, the system allowing for replication and translation are living cells.

In another embodiment, the system allowing for replication and translation are living human cells.

In another embodiment, the system allowing for replication and translation is a prokaryotic or eukaryotic cell-free extract containing endogenous ribosomes, endogenous transfer RNA and endogenous amino acids.

In another embodiment, the modulator is an inhibitor.

In another embodiment, the tRNA-related condition is cancer, in particular metastatic cancer.

Another aspect of the invention is a kit for the detection of a modulator of a tRNA-related condition comprising one or more recombinant DNA constructs comprising
i. A DNA oligonucleotide sequence encoding a first luminescence-based reporter gene operatively linked to a first promoter,
ii. A DNA oligonucleotide sequence encoding a second luminescence-based reporter gene operatively linked to a second promoter,
wherein the recombinant DNA constructs comprise 3 to 10 in frame repetitions of a first codon upstream of the first luminescence-based reporter gene;
wherein the first codon is a tRNA modification-sensitive codon,
wherein the recombinant DNA constructs further comprises 3 to 10 in frame repetitions of a second codon upstream of the second luminescence-based reporter gene, and
wherein the second codon is the synonymous codon of the first codon.

Another aspect of the invention is the use of one or more recombinant DNA constructs comprising
i. A DNA oligonucleotide sequence encoding a first luminescence-based reporter gene operatively linked to a first promoter;
ii. A DNA oligonucleotide sequence encoding a second luminescence-based reporter gene operatively linked to a second promoter,
for the detection of a modulator of a tRNA-related condition;
wherein the recombinant DNA constructs comprise 3 to 10 in frame repetitions of a first codon upstream of the first luminescence-based reporter gene;
wherein the first codon is a tRNA modification-sensitive;
wherein the recombinant DNA constructs further comprises 3 to 10 in frame repetitions of a second codon upstream of the second luminescence-based reporter gene; and
wherein the second codon is the synonymous codon of the first codon.

### DETAILED DESCRIPTION OF THE INVENTION

tRNA Modification-Sensitive Codons

A transfer ribonucleic acid, hereinafter referred to as tRNA, is a molecule composed of typically 70 to 90 ribonucleotides. It serves as the link between the nucleotide sequence of nucleic acids and the amino acid sequence of proteins. Its primary function is carrying a specific amino acid to the ribosome as directed by the three-nucleotide codon sequence in messenger RNA, the so-called mRNA.

t-RNA modification-sensitive codon is meant to comprise any codon susceptible of the modification of the cognate tRNA.

Biological phenotypes associated with modulation of a tRNA modification have been associated with decoding of specific codons during translation elongation. Preferred tRNA modifications comprise modifications of the anti-codon loop of tRNAs. In a particularly preferred embodiment, the tRNA modification is the wobble uridine tRNA modification. The mcm5s2 modification occurring on the U34 position of certain tRNAs is the best studied example of a decoding modulator of the specific codons AAA, GAA and CAA, hereinafter referred to as the U34-codons. Other examples of tRNA modifications in the anti-codon loop and their corresponding tRNA modification-sensitive codons are listed in the Table 1 below.

TABLE 1
tRNA modification-sensitive codons.

| tRNA- | | t-RNA modification- |
|---|---|---|
| anticodon | Detected modification(s) on oligonucleotide(s) | sensitive codon |
| Ala-GGC | CA[Um]UGp (T1), UGGCUGp (MC1) | GCC |
| Ala-UGC | CU[cnm5U]GCp (Cus), CAAGp (T1) | GCA |
| Arg-GCG | GGCCUGCp (A), GCGGAGCCp (A) | CGC |
| Arg-UCG | [cnm5U]CG[imG-14]AUp (A) | CGA |
| Arg-UCU | CCU[cnm5U]CU[t6A]AGp (T1), CCU[cnm5s2U]CU[t6A]AGp (T1) | AGA |
| Asn-GUU | GUU[t6A]AUp (A) | AAC |
| Asp-GUC | GGGACUGUp (A), UCACUCCCGp (T1) | GAC |
| Cys-GCA | UGCA[m1G]AUCCGCCp (MC1), UGCA[imG-14]AUCCGCCp (MC1), CA[imG- | UGC |
| | 14]AUCCGp (T1) | |
| Gln-UUG | U[mnm5U]UG[m1G]ACCCCp (Cus), U[mnm5s2U]UG[m1G]ACCCCp (Cus) | CAA |
| Glu-UUC | CCU[mnm5s2U]UC[m1G]AGp (T1) | GAA |
| Gly-GCC | GGGC[Cm]UGCp (A), CCACGp (T1) | GGC |
| Gly-UCC | CCU[mnm5U]CCAAGp (T1) | GGA |
| His-GUG | GUG[m1G]AUCp (A) | CAC |
| Ile-GAU | GAU[t6A]ACp (A), GAU[hn6A]ACp (A) | AUC |
| Ile-CAU | CU[C+]AU[hn6A]ACCGp (T1) | AUG |
| Leu-GAG | GAGGGUCUp (A) | CUC |
| Leu-UAA | [cnm5U]AA[m1G]AUCp (A), [cnm5U]AA[imG]AUCp (A), [cnm5s2U]AA[m1G]AUCp (A) | UUA |
| Leu-UAG | CC[cnm5s2U]AGpb (T1), [m1G]ACCCAGp (T1) | CUA |
| Lys-UUU | [Cm]U[cnm5s2U]UU[t6A]ACCAGp (T1), [Cm]U[cnm5s2U]UU[hn6A]ACCAGp (T1) | AAA |
| Met-CAU1 | CU[Cm]AU[hn6A]ACCGp (T1), CU[Cm]AU[ms2hn6A]ACCGp (T1) | AUG |
| Met-CAU2 | CUCAUAACCCGp (T1) | AUG |
| Phe-GAA | AA[m1G]AUCCAGp (T1), GAA[m1G]AUp (A), GAA[imG]AUp (A) | UUC |
| Pro-GGG | GGG[m1G]GGCp (A) | CCC |
| Pro-UGG | AUU[cnm5U]Gp (T1), [m1G]AUCCUGp (T1) | CCA |
| Ser-GCU | GGGACUGCp (A), CU[hn6A]AUCCCAUUGp (T1) | AGC |
| Ser-GGA | GGA[m1G]AUCpb (A), GGA[imG]AUCpb (A) | UCC |
| Ser-UGA | U[cnm5s2U]GA[m1G]AUCp (A) | UCA |
| Thr-GGU | GGU[hn6A]AGCp (A) | ACC |
| Thr-UGU | GACU[cnm5U]GUp (A), U[hn6A]AUCAGp (T1) | ACA |
| Trp-CCA | A[s2C]UCCA[m1G]AUCCCUGp (T1) | UGG |
| Tyr-GUA | GUA[imG-14]AUp (A) | UAC |
| Val-CAC | CCCUCACAAGp (T1) | GUG |
| Val-GAC | CC[Cm]UGpb (T1), ACACGp (T1) | GUC |
| Val-UAC | CCU[cnm5U]ACp (A), CCCU[cnm5U]ACAAGp (T1) | GUA |

### Constructs, Target Codons and Synonymous Codons

The constructs of the present invention may be any recombinant DNA construct capable of replication, in particular plasmids. In one embodiment, the plasmid is bi-cistronic.

In another embodiment, the plasmid comprises a single ORF. Bi-cistronic plasmids are preferred.

In a preferred embodiment, the constructs comprise an equal number or repetitions of thee target codon and of the synonymous target codon.

The number of repetitions is chosen in a way as to ensure a stability of the translation system. Stable translation systems may usually be obtained between 3 and 10 repetitions of the first or of the second codon.

### Luminescence-Based Reporter Gene

The present assay comprises a first and a second luminescence-based reporter gene. The first and the second luminescence-based reporter genes emit two different luminescence-based signals so as to allow for differentiation between the activation and level of activation of the first and the second luminescence-based reporter gene in a luminescence reader.

In one embodiment the luminescence-based reporter gene is a luminescence-based reporter gene.

In a preferred embodiment, the first and the second luminescence based reporter genes are the dual reporter system of Luciferase-Renilla.

In another embodiment, a single luminescence-based reporter gene system is optimized by adding PEST sequences to luciferase reporters.

### Ribosome-Containing Medium or Host Cell

The assay of the present invention may be performed in any system that allows for translation.

In one embodiment, the assay is performed in a living host cell, in particular a human living host cell.

In another embodiment, the assay is performed in prokaryotic or eukaryotic cell-free extract from a living organism containing endogenous ribosomes, endogenous transfer RNA and endogenous amino acids.

### Transduction System

The host cells may be stably transduced.

In one embodiment, the stable transduction system is the lentiviral system.

In a preferred embodiment, the host cells are stably transduced with pLV-Puro-hPGK and 1:3 Mirus transfecting agent as described in Close et al, Mol Cell 2006.

In another embodiment, the host cells are transiently transduced.

In a preferred embodiment, the host cells are transiently transduced with lipofectamine 2000 or any lipid based transfectant reagent (transfectine, dharmafect, turbofectin, lentitran).

In one embodiment, 0.2 μg plasmid and 0.5 μl lipid based transfectant reagent are applied.

### Cell Culture and Cell-Based Assay

The transduced cells may be cultured in 48 to 96 well plates. Each well plate may comprise 10.000 to 30.000 cells.

In one embodiment, the transduced cells are treated with the candidate tRNA modification for one hour or more, preferably for two hours or more, even more preferably for 4 hours or more.

Treatment means any exposure, dosage or administration of the candidate tRNA modification to transduced cells so as to produce a translation modulation effect. A skilled person, through routine essays, may determine a suitable amount of treatment.

In another embodiment, the transduced cells are treated with the candidate tRNA modification for 48 hours or less, preferably for 24 hours or less, even more preferably for 12 hours or less.

In preferred embodiment, transduced cells are treated with the candidate tRNA modification for 2 to 4 hours, or for 4 two 8 hours.

In one embodiment, the treatment is repeated three times to show repeatability and statistical relevance of the translation modulation.

The luminescence signal may be produced according to procedure known to the skilled person.

In the preferred embodiment, the luminescence-based reporter system is the Nano-Glo® Dual-Luciferase® Reporter (NanoDLR™) Assay System commercially available from Promega. The medium may be removed and 40 microliters of complete cell culture medium may be added. The plate may be equilibrated at room temperature for 15 min. 40 microliters of ONE-Glo™ EX Reagent may be added for each well. The plates may be mixed and shaked and protected from light for 10 minutes. Then the luminescence may be read. 40 microliters of NanoDLR™ Stop and GloR Reagent may be freshly prepared and added to each well. The plates may be mixed and shaked and protected from light for 10 minutes. The luminescence may be read by any luminescence reader, preferably. The luminescence may be analyzed by calculating the Nanoluc/Firefly ratio.

Candidate tRNA Modification Modulator

A candidate tRNA modification modulator may be any known or new chemical compound, small molecule, protein or other a composition comprising one or more chemical compounds, small molecule or proteins. In general, a candidate tRNA modification modulator is not yet known to have a modulating effect on codon translation.

Modulation means any impact on translation or modification of translation caused by directly or indirectly by candidate tRNA modification as compared to a system that has not been exposed to the candidate tRNA modification. Modulation includes decrease, increase, stabilization or control.

In one embodiment, the tRNA modification modulator decreases or inhibits translation of the first codon, wherein the translation of the first codon requires a tRNA modification, in particular a cancer-related tRNA modification.

In a preferred embodiment, the candidate tRNA modification modulator decreases or inhibits translation of the tRNA modification-sensitive codons, in particular the U34 codons.

### Application

In a preferred embodiment, a library of dual luciferase constructs with combination of synonymous codons is generated to identify compounds with direct activity on codon-decoding. No expensive machinery or advance bioinformatics analysis is required. Preferably, luminescence is measured by TECAN-like instruments.

In another embodiment, the modulator effect on tRNA translation of any compound, diet, or biological organism that affects codon-dependent translation optimization may be assessed. Possible application fields include pharma, agriculture, or biodiversity studies.

### Advantages

Identifying modulators of mRNA translation rate requires to rapidly assess their impact on codon-dependent translation in biological relevant settings. The cell-based assay of the present invention allows to test the effects on codon-dependent translation of candidate tRNA modification compounds in a fast, cost-effective and reproducible manner without the need for extensive computational and experimental knowhow, resources and sample size. In particular, no ribosomal sequencing apparatus and associated computing is required.

Importantly, the results of the present cell-based assays are available within 2 to 4 hours. Ribosome sequencing takes in general 4 to 10 days.

### SHORT DESCRIPTION OF THE DRAWINGS

FIG. 1a shows dual luciferase constructs encoding six in frame repetitions of U34 codons (xAA) upstream of NanoLuc and six in frame repetitions of the corresponding synonymous codon (xAG) upstream of FireflyLuc.

FIGS. 1b-d shows a strong decrease in NanoLuc/Firefly ratio in MDA-MB231 human breast cancer cells after exposure of 48 hours to IPTG, Isopropyl β-D-1-thiogalactopyranoside for inducible depletion of U34-enzymes by shRNA (right panels, confirm depletion of the U34-enzymes after IPTG administration).

FIG. 1e-shows that no effect was obtained in recombinant DNA constructs lacking the codon repetitions in MDA-MB231 breast cancer cells (FIG. 1e; right panel, confirms depletion of the U34-enzymes after IPTG administration).

FIG. 1f shows the increased luciferase specific ratio through the tRNA modification modulator insulin (250 nM for 24 hours) in M395 human primary melanoma cells.

FIGS. 1g-h show that the dual PI3K/mTOR inhibitor NVP-BEZ235 and the mTOR inhibitor PP242 decreased luciferase specific signal in a time and concentration-dependent manner in A549 human lung cells

### EXAMPLES

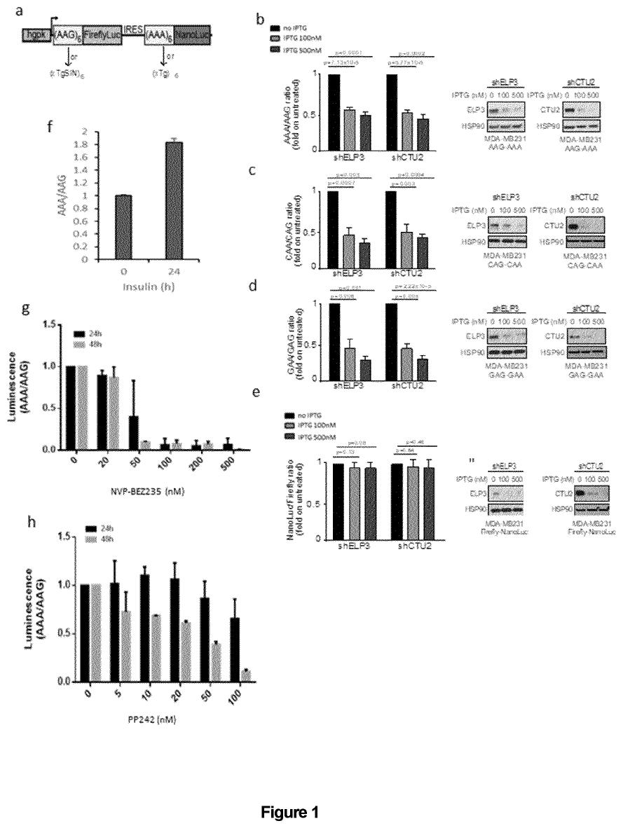

Dual luciferase constructs encoding six in frame repetitions of U34 codons (xAA) upstream of NanoLuc and six in frame repetitions of the corresponding synonymous codon (xAG), whose decoding does not require U34-tRNA modification, upstream of FireflyLuc were provided (FIG. 1a).

Cells (MDA-MB231, M395 or A549) were stably transduced by the lentiviral system (pLV-Puro-hPGK) and 1:3 Mirus transfecting agent [Close et al, Mol Cell 2006].

Cells were culture in a suitable culture system in the presence of the candidate tRNA modification modulator.

Signals were detected by any luminescence reader, for example the TECAN Infinite M2000 Pro. Read time can be between 1 and 3 seconds with automatic attenuation.

IPTG inducible depletion of the U34 enzymes, ELP3 or CTU2, strongly decreased NanoLuc activity but did not impact FireflyLuc activity (FIGS. 1b-d, 48 hours IPTG induction). No effect was seen using a construct lacking the codon repetitions (FIG. 1e). Same experimental settings were used to test the translation effects of known activators or inhibitors of the U34 enzymes [Rapino et al., Nature 2018]. The inducer insulin increased luciferase specific ratio (FIG. 1f), on the contrary the dual PI3K/mTOR inhibitor NVP-BEZ235 and the mTOR inhibitor PP242 decreased luciferase specific signal in a time and concentration dependent manner (FIG. 1g-h).

TABLE 2
Translation of abbreviations and of the
English expressions used in the drawings:

| | |
|---|---|
| English expression | Translation |
| FireflyLuc | FireflyLuc |
| NanoLuc | NanoLuc |
| Fold on untreated | |
| Luminescence | |
| xTgSIN = any synonymous codons of the | |
| tRNA modification-sensitive codons | |
| xTg = tRNA modification-sensitive codons | |
| nM = nanomolar | |
| h = hours | |
| tRNA modification-sensitive codons = | |
| codons that require modified tRNA to be | |
| correctly/efficiently translated. | |

1. An assay for detecting a modulator of a tRNA-related condition, comprising the steps of:
a. Providing one or more recombinant DNA constructs comprising
i. A DNA oligonucleotide sequence encoding a first luminescence-based reporter gene operatively linked to a first promoter;
ii. A DNA oligonucleotide sequence encoding a second luminescence-based reporter gene operatively linked to a second promoter;
b. Introducing the one or more recombinant DNA constructs of step a) into a system so as to allow for replication and translation in order to obtain a transduced system;
c. Exposing the transduced system of step b) to one or more candidate tRNA modification modulators to obtain a modulated system;
d. Inducing the luminescence emission of the modulated system of step c) to obtain an induced system;
e. Measuring the luminescence emission of the first and of the second luminescence-based reporter genes in the induced system of step d);
f. Correlating the signal measured in step e) to the one or more candidate tRNA modification modulators; and
g. Optionally selecting a modulator of a tRNA-related condition from the one or more candidate tRNA modification modulators;
Wherein the recombinant DNA constructs comprise 3 to 10 in frame repetitions of a first codon upstream of the first luminescence-based reporter gene;
Wherein the first codon is a tRNA modification-sensitive codon;
Wherein the recombinant DNA constructs further comprises 3 to 10 in frame repetitions of a second codon upstream of the second luminescence-based reporter gene,
Wherein the second codon is the synonymous codon of the first codon.

2. The assay of claim 1, wherein the tRNA modification-sensitive codon is selected from the group consisting of
a. GCC
b. GCA
c. CGC
d. CGA
e. AGA
f. AAC
g. GAC
h. UGC
i. CAA
j. GAA
k. GGC
l. GGA
m. CAC
n. AUC
o. AUG
p. CUC
q. UUA
r. CUA
s. AAA
t. AUG
u. AUG
v. UUC
w. CCC
x. CCA
y. AGC
z. UCC
aa. UCA
bb. ACC
cc. ACA
dd. UGG
ee. UAC
ff. GUG
gg. GUC
hh. GUA

3. The assay of claim 1, wherein the t-RNA modification-sensitive codon is selected from the group consisting of AAA, GAA, and CAA.

4. The assay of claim 1, wherein the second codon is selected from the group consisting of AAG, GAG and CAG.

5. The assay of claim 1, wherein the recombinant DNA constructs comprise 4 to 8 repetitions of the first and of the second codon.

6. The assay of claim 1, wherein the recombinant DNA constructs comprise 6 in frame repetitions of the target codons and of the corresponding synonymous codon.

7. The assay of claim 1, wherein the first or the second reporter genes are fluorescence-based reporter genes

8. The assay of claim 1, wherein the luminescence-based based reporter genes are Nanoluc, Firefly; Nanoluc-PEST or Firefly-PEST.

9. The assay of claim 1, wherein the system allowing for replication and translation are living cells.

10. The assay of claim 1, wherein the system allowing for replication and translation are living human cells.

11. The assay of claim 1, wherein the system allowing for replication and translation is a prokaryotic or eukaryotic cell-free extract containing endogenous ribosomes, endogenous transfer RNA and endogenous amino acids.

12. The assay of claim 1, wherein the tRNA modification modulator is an inhibitor.

13. The assay of claim 1, wherein the tRNA-related condition is cancer, in particular metastatic cancer.

14. A kit for the detection of a modulator of a tRNA-related condition the comprising one or more vials containing cells comprising one or more recombinant DNA constructs comprising
i. A DNA oligonucleotide sequence encoding a first luminescence-based reporter gene operatively linked to a first promoter;
ii. A DNA oligonucleotide sequence encoding a second luminescence-based reporter gene operatively linked to a second promoter;
Wherein the recombinant DNA constructs comprise 3 to 10 in frame repetitions of a first codon upstream of the first luminescence-based reporter gene;
Wherein the first codon is a modification-sensitive codon;
Wherein the recombinant DNA constructs further comprises 3 to 10 in frame repetitions of a second codon upstream of the second luminescence-based reporter gene, and
Wherein the second codon is the synonymous codon of the first codon.

15. Use of one or more recombinant DNA constructs comprising
i. A DNA oligonucleotide sequence encoding a first luminescence-based reporter gene operatively linked to a first promoter;
ii. A DNA oligonucleotide sequence encoding a second luminescence-based reporter gene operatively linked to a second promoter;
for the detection of a modulator of a tRNA-related condition;
Wherein the recombinant DNA constructs comprise 3 to 10 in frame repetitions of a first codon upstream of the first luminescence-based reporter gene;
Wherein the first codon is a tRNA modification-sensitive codon;
Wherein the recombinant DNA constructs further comprises 3 to 10 in frame repetitions of a second codon upstream of the second luminescence-based reporter gene; and
Wherein the second codon is the synonymous codon of the first codon.
